(19) United States
(12) Patent Application Publication
(10) Pub. No.: US 2010/0255467 A1
(43) Pub. Date: Oct. 7, 2010

(54) ASSAYS FOR DETERMINING EXPOSURE TO MULTIWALLED CARBON NANOTUBES

(75) Inventors: Kevin D. Houston, Las Cruces, NM (US); Min Sung Park, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: LOS ALAMOS NATIONAL SECURITY, LLC, Los Alamos, NM (US)

(21) Appl. No.: 12/507,657

(22) Filed: Jul. 22, 2009

Related U.S. Application Data
(60) Provisional application No. 61/135,690, filed on Jul. 22, 2008.

(51) Int. Cl. C12Q 1/68; G01N 33/53

(52) U.S. Cl. 435/6; 435/7.21; 435/7.24

(57) ABSTRACT

Assays useful in determining exposure to multi-walled carbon nanotubes (MWCNTs) are provided. In one aspect, the MWCNT exposure assays operate by detecting a significant increase in the expression levels and/or status of certain cytokines shown to be responsive to MWCNT exposure.

### RELATED APPLICATIONS

This patent application claims the benefit of the filing date of U.S. Provisional patent application No. 61/135,690 filed Jul. 22, 2008 under 35 U.S.C. 119(e).

### STATEMENT AS TO RIGHTS TO INVENTIONS MADE UNDER FEDERALLY SPONSORED RESEARCH OR DEVELOPMENT

This invention was made with government support under Contract No. DE-AC52-06 NA 25396 awarded by the U.S. Department of Energy. The government has certain rights in the invention.

### BACKGROUND OF THE INVENTION

The potential for increased human exposure to engineered nanotechnology materials and derivative products is high due to ongoing development of nanomaterials aimed at applications ranging from biomedical imaging to solar energy capture¹⁻⁴. One such nanomaterial, carbon-based nanotubes, have unique properties that make them ideal for biomedical applications such as drug delivery⁵. In order to benefit from the myriad of proposed uses of carbon-based nanotubes, it is imperative that potential adverse effects on human health are identified prior to their incorporation into both household and specialized products. Suitable strategies for identifying potential adverse human health effects, including inflammation and undesirable bioactivities such as activation of specific cell signal transduction pathways, are not currently available.

In initial attempts to determine if exposure to engineered carbon nanotubes results in adverse human health effects, many research groups have evaluated the cytotoxicity of these materials using various model systems and biological endpoints. However, for both single-walled and multi-walled carbon nanotubes, potential bioactivity and cytotoxicity have not been clearly established, and results from different groups are often contradictory. Some reports provide evidence that multi-walled carbon nanotubes (MWCNT) are toxic to certain cell lines, while other groups report that MWCNTs are nontoxic⁶⁻¹⁰. Still other reports indicate that the potential for MWCNT cytotoxicity is dependent on residual catalyst or the size of the nanotube with regard to length^{11, 12}. To further complicate the issue, an MTT assay used to evaluate cytotoxicity was later shown ineffective due to unforeseen chemical interactions between the carbon nanotubes and the reporter precipitate¹³. Also, observed cytotoxic effects may be a function of the way the treatment was prepared (e.g., sonication or the use of a surfactant)⁶.

Very recently, researchers at the MRC/University of Edinburg and colleagues reported that exposing the mesothelial lining of the body cavity of mice, a surrogate for the mesothelial lining of the chest cavity, to long MWCNTs results in asbestos-like, length-dependent, pathogenic behavior, including inflammation and the formation of lesions known as granulomas (Poland et al., 2008, Nature Nanotechnology 3, 423-428). The effect was not observed with short MWCNTs.

Together, however, the current data regarding the cytotoxic effect and potential for bioactivity associated with carbon nanotubes exposure is not conclusive and biomarkers for exposure have not been reported.

While a great effort to determine the cytotoxic potential of carbon nanotubes is ongoing, it is important to understand the potential for nanotube bioactivity as well as to identify biomarker profiles associated with exposure to carbon nanotubes. An example of a potential carbon-based nanotube bioactivity is the activation of a specific cell signal transduction pathway upon receptor binding. Such bioactivity, if not identified, could lead to unforeseen negative human health consequences. Furthermore, the identification of biomarker profiles would facilitate the development of screening tools to evaluate the safety of exposure to carbon-based nanomaterials.

### SUMMARY OF THE INVENTION

The invention provides assays useful in determining an individual's exposure to MWCNTs, which function by detecting a significant increase in the mRNA or protein expression of biomarker profiles associated with MWCNT exposure. In one aspect, the invention's MWCNT exposure assays operate by detecting a significant increase in the expression levels (or status) across one or more panels of cytokines shown to be responsive to MWCNT exposure. The MWCNT exposure assays of the invention measure cytokine mRNA or protein expression in a test biological sample relative to expression levels in a corresponding normal sample.

In one embodiment, an assay for evaluating MWCNT exposure in an individual, comprises determining the expression levels of cytokines in a core cytokine panel, consisting of IL-6, IL-9, IFNγ, TNFα and GM-CSF, in a test biological sample obtained from the individual, and comparing the expression levels so determined to the expression levels in a corresponding normal sample. The presence of significantly elevated expression levels of each of the cytokines IL-6, IL-9, IFNγ, TNFα and GM-CSF in the test sample relative to the normal sample provides an indication of MWCNT exposure.

In another embodiment, the expression levels of a wider cytokine panel consisting of the core panel, above, plus one or more of the cytokines IL-1α, IL2, IL3, IFNβ and CCL5, are evaluated in both the test and normal samples. The presence of significantly elevated expression levels of each of the cytokines in the core panel, plus significantly elevated expression levels in one or more of the additional cytokines IL-1a, IL2, IL3, IFNβ and CCL5, in the test sample relative to the normal sample, provides an indication (and/or confirmation, relative to the assay with the core cytokine panel) of MWCNT exposure.

Another embodiment provides an assay for evaluating MWCNT exposure in an individual, comprising detecting the presence of phosphorylated IRF3 protein in a biological sample from the individual. The presence of phosphorylated IRF3 protein in the biological sample provides an indication of exposure to MWCNTs.

Yet another embodiment combines an assay with the core cytokine panel, or an assay with the core cytokine panel and one or more of the additional cytokines IL-1α, IL2, IL3, IFNβ and CCL5, with an assay for detecting the presence of phosphorylated IRF3 protein in the test sample. Detection of phosphorylated IRF3 protein in the test sample provides an indication (and/or confirmation) of MWCNT exposure.

Expression levels of the cytokines IL-1α, IL2, IL3, IL-6, IL-9, IFNβ, IFNγ, TNFα, GM-CSF and CCL5 may be determined at the protein level or at the nucleic acid (mRNA) level, using standard methods known in the art. Detection of phosphorylated IRF3 is determined at the protein level.

Biological test samples may be any sample which would be expected to contain cytokine protein or cytokine mRNA, including without limitation peripheral blood, serum, and cell fractions thereof.

The invention also provides assays designed to evaluate a biological activity of a MWCNT preparation. In one embodiment, the invention provides an in vitro assay for evaluating whether a MWCNT preparation is capable of initiating the IRF3-mediated signal transduction pathway of the innate immune system in mammalian cells, comprising exposing cultured mammalian cells to the MWCNT preparation, extracting total protein from the cells, and detecting phosphorylated IRF3 protein therein. The detection of phosphorylated IRF3 protein provides an indication that the MWCNT preparation is capable of initiating the IRF3-mediated signal transduction pathway of the innate immune system. Typically, the assay will be an immunoassay, in which an antibody specific for phosphorylated IRF3 is employed. Any mammalian cells in which IRF3 can be activated may be used, including, for example, monocytes, macrophages, lymphocytes and other leukocytes.

### BRIEF DESCRIPTION OF THE DRAWINGS

FIG. 1. Fold induction of specific chemokines/cytokines in MWCNT exposed cells. (a) RAW264.7 cells exposed to sonicated cell growth media (SM), 50 μg/mL, or 500 μg/mL MWCNT suspensions in sonicated cell growth media. (b) RAW264.7 cells treated with SM, 25 μg/ml polyinosinic:polycytidylic acid (Poly I:C), or 100 ng/mL lipopolysaccaride (LPS) in SM. Data are representative of three independent experiments and error bars represent standard error of the mean (SEM).

FIG. 2. TEM images of the nanotubes used in cell exposure experiments. (a) MWCNT obtained from BuckyUSA, (b) aMWCNT obtained from NanoAmor, (c) sMWCNT obtained from NanoAmor, and d) SWCNT obtained from NanoAmor.

FIG. 3. Normalized fold induction of specific chemokines/cytokines in nanotube or fullerene exposed cells. (a) RAW264.7 cells exposed to sonicated cell growth media (SM) or 500 μg/mL suspensions of MWCNT, aMWCNT, or sMWCNT in sonicated cell growth media. (b) RAW264.7 cells exposed to sonicated cell growth media (SM) or 500 μg/mL suspensions of SWCNT or fullerene (C₆₀) in cell growth media. Data are representative of three independent experiments and error bars represent standard error of the mean (SEM).

FIG. 4. Interferon beta (IFNβ) concentration in cell culture supernatants from RAW264.7 cells exposed to sonicated cell growth media (SM) 25 μg/mL Poly I:C, 5 μg/mL, 50 μg/mL or 500 μg/mL MWCNT suspension in cell growth media. Data are representative of three independent experiments and error bars represent standard error of the mean (SEM). * denotes statistical significance from SM treatment (P<0.05 calculated using student's T-test).

FIG. 5. Immunoblot analysis of interferon regulatory factor 3 (IRF3) protein expression and the accumulation of phosphorylated IRF3 (p-IRF3) upon MWCNT (BuckyUSA) exposure in RAW264.7 cells. Data are representative of three independent experiments.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial, difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “nucleic acid” refers to deoxyribonucleotides or ribonucleotides and polymers thereof (“polynucleotides”) in either single- or double-stranded form. Unless specifically limited, the term “polynucleotide” encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The term nucleic acid is used interchangeably with gene, cDNA, and mRNA encoded by a gene.

As used herein, the term “cytokine” refers to a group of signaling proteins and glycoproteins that are produced by a wide variety of hematopoietic and non-hematopoietic cell types, and includes chemotactic cytokines or chemokines. The cytokine family consists mainly of smaller, water-soluble proteins and glycoproteins with a mass of between 8 and 30 kDa. The terms “chemokine” and “chemotactic cytokine” refers to members of a specific class of cytokines that mediate chemoattraction (chemotaxis) between cells.

Standard cytokine abbreviations are used herein and include IL-1α and IL-1β (interleukins 1α and 1β), IL-2 (interleukin 2), IL-3 (interleukin 3), IL-4 (interleukin 4), IL-5 (interleukin 5), IL-6 (interleukin 6), IL-9 (interleukin 9), IL-10 (interleukin 10), IL-12 (interleukin 12), IFNβ (Beta-Interferon), IFNγ (Gamma-Interferon), GM-CFS (Granulocyte Macrophage-Stimulating Factor) and TNFα (Tumor Necrosis Factor α). The term “RANTES” refers to the chemokine CCL5, and the two terms are used interchangeably herein.

### Assays for Evaluating MWCNT Exposure:

Determining the status of MWCNT-induced cytokine expression in an individual may be used to diagnose and possibly gauge the level of exposure to MWCNTs, monitor remedial efforts designed to mitigate exposure, and/or provide prognostic information useful in defining appropriate therapeutic options. The invention provides methods and assays for determining MWCNT-induced cytokine expression status as a means for providing such diagnostic information.

In one aspect, the invention provides assays useful in determining exposure to MWCNTs in an individual, comprising detecting a significant increase in the expression status (expression level) across one or more panels of cytokines shown to be responsive to MWCNT exposure. The MWCNT exposure assays of the invention measure cytokine mRNA or protein expression in a test biological sample relative to expression levels in a corresponding normal sample. Such a “normal” reference sample may be obtained from an individual known not to have any exposure to MWCNTs, including for example, individuals sampled prior to becoming engaged in an activity which might result in exposure, such as employment at a MWCNT production facility.

In one embodiment, the expression status of a 5-member cytokine panel is evaluated in a biological sample obtained from an individual to be screened for possible exposure to MWCNTs. Typically, the biological sample will be a peripheral blood or plasma sample, or a particular cell preparation extracted therefrom, such as a white blood cell (leukocyte) fraction preparation. This “core cytokine panel”, as it is referred to herein, includes IL-6, IL-9, IFNγ, TNFα and GM-CFS. Applicants' research shows that macrophages express significantly increased levels of these five cytokines when exposed to various preparations of MWCNTs (see Examples 1 and 2, infra). Moreover, this MWCNT biomarker profile appears to be specific for MWCNTs, as no similar profile is seen in parallel exposure studies with both single walled CNTs or fullerene suspensions (see Example 3, infra). Therefore, detecting a significant increase in the expression status of the members of the core cytokine panel provides an indication of the individual's exposure to MWCNTs.

Any significant increase in the expression status of all of the members of the core cytokine panel may provide an indication of exposure to MWCNTs, particularly unacceptably high level exposure. Generally, exposure to high levels of MWCNTs is correlated with an increase of at least one fold over normal. However, expression level increases may range across the core cytokine panel from one to seven-fold compared to expression levels in normal samples. For example, as demonstrated in Example 1, infra, high level exposure can result in up to about a seven-fold induction in one of the cytokines in the core panel, TNFα.

The level of increased expression across the members of the core cytokine panel may correlate with the degree, duration and/or severity of exposure. Initial studies indicate that progressively higher levels of MWCNTs result in higher fold expression levels (see Examples, infra). Accordingly, the MWCNT exposure assays of the invention may provide qualitative information about exposure levels, and may similarly be applied to post-exposure monitoring programs designed to evaluate the attenuation of MWCNT exposure levels in individuals who are deemed to have been unacceptably exposed.

In a related embodiment, additional cytokines are also evaluated along with those of the core cytokine panel. Because MWCNT exposure also induces upregulated expression of the cytokines IL-1α, IL2 and IL3, as well as IFNβ and the chemotactic cytokine CCL5 (or, RANTES), these cytokines may provide additional evidence of MWCNT exposure across a wider biomarker profile. Accordingly, the invention also provides assays in which both the core cytokine panel and one or more of the foregoing additional cytokines are evaluated for expression status in a test biological sample from an individual to be screened for MWCNT exposure, wherein a significant difference in the expression status in the test sample relative to a normal reference or sample across all or most of the cytokines used in the wider panel provides an indication of the individual's exposure to MWCNTs.

In a particular embodiment, the expression status of extracellular IFNβ is measured, in combination with evaluating the expression status across the core cytokine panel. As demonstrated by the results obtained in Example 4, infra, exposure to MWCNTs in macrophage-like cells results in increased levels of secreted IFNβ. Moreover, the effect is dose dependent, with higher concentrations of MWCNTs progressively inducing higher levels of IFNβ expression. Thus, in combination with the core cytokine panel assay of the invention, the IFNβ assay not only may provide confirmation of MWCNT exposure, but also may be particularly useful in gauging the level, duration and/or overall severity of an individual's exposure to MWCNTs. Similarly, following the implementation of an exposure mitigation plan, continual monitoring of IFNβ levels may be useful to assess falling exposure levels and/or MWCNT clearance form the individual.

In another, related aspect of the invention, assays which detect the activation of the interferon regulatory factor 3 (IRF3)-mediated signal transduction pathway of the innate immune response are provided. Applicants have discovered that MWCNT exposure also leads to accumulation of intracellular phosphorylated IRF3, the activated form of this transcription factor, in a dose dependent manner. IFNβ and CCL5, both of which are upregulated upon MWCNT exposure in macrophage-like cells (see Examples 1 and 2, infra), are associated with an innate immune response to viral infection and are produced via activation of the IRF3 transcription factor. IRF3 is activated upon C-terminal phosphorylation by TBK-1 and/or IKKi, and, when phosphorylated, IRF3 homodimers form and translocate to the nucleus where they enhance the transcription of IFNβ and CCL5²¹⁻²³. IRF3 activation can be observed via the accumulation of the phosphorylated species of IRF3 (p-IRF3) in whole cell protein extracts (See Example 5, infra).

Thus, in one embodiment of this aspect of the invention, the presence of intracellular activated IRF3 (p-IRF3) is evaluated in a biological sample obtained from an individual to be screened for possible exposure to MWCNTs. Typically, the biological sample will be a peripheral blood or plasma sample, or a particular cell preparation extracted therefrom, such as a white blood cell (leukocyte) fraction preparation. Detecting intracellular p-IRF3 provides an indication of the individual's exposure to MWCNTs. See Example 5, infra.

Because there are various ways in which MWCNTs may be prepared (e.g., catalytic chemical vapor deposition vs. arc discharge), as well as variability in sizes and purification levels, cytokine expression profiles generated from samples of individuals exposed to MWCNTs may differ somewhat according to both the MWCNT type, source and fabrication process. However, this may translate into an ability to determine fine differences between MWCNT biomarker profiles, thereby enabling the generation of MWCNT exposure assays that not only provide an indication of exposure, in general, but also an indication (particular signature) of a specific type of MWCNT.

Methods for quantifying the expression of cytokine mRNA or protein use standard nucleic acid and protein detection and quantification technologies well known in the art.

Expression status may be evaluated immunologically, using various immunological assay formats well known in the art, including but not limited to various types of immunoprecipitation, agglutination, complement fixation, radioimmunoassays, Western Blot, enzyme-linked immunosorbent and immunofluorescent assays, enzyme-linked assays, ELISA immunoassays, immunohistochemical analysis and the like. Immunological assays for cytokines which include the MWCNT-inducible cytokines evaluated in the assays of the invention are well known and widely available commercially.

Alternatively, expression status may be evaluated at the mRNA level, by obtaining a peripheral blood or plasma sample, or a fraction thereof which includes a MWCNT-responsive cell population, such as a macrophage or helper T lymphocyte cell population, and assaying for mRNA expression levels across the desired cytokine panel(s) in the population. Numerous assay methods for quantitatively measuring mRNA levels are known in the art, including without limitation RT-PCR assays, TaqMan® gene expression assays (e.g., Applied Biosystems), as well as various other amplification assays such as NASBA, SISBA, branched DNA assays, helicase dependent amplification, strand displacement amplification, and the like.

### MWCNT Biological Activity Screening Assays:

In another aspect of the invention, assays designed to evaluate a biological activity of a MWCNT preparation are provided. Applicants have determined that MWCNT preparations induce a particular cytokine expression profile not induced by other types of carbon containing structures, including fullerene and single wall CNT structures. Accordingly, the specific cytokine expression profiles disclosed herein may be used to screen MWCNT preparations for their capacity to induce upregulated cytokine expression, using an appropriate cell-based assay system, such as the cell-based assay employed and described in Examples 1-5, infra. MWCNT preparations may be screened with the assays of the invention in order to determine their likely impact on individuals exposed to such preparations or to downstream products fabricated with such preparations. Various modifications to such preparations my similarly be evaluated for the ability to attenuate undesirable biological activities.

In an exemplary embodiment, the invention provides an in vitro assay (e.g., immunoassay) which measures induction of intracellular activated IRF3 (p-IRF3) in responsive cells (e.g., macrophages) treated with a MWCNT preparation. Typically, responsive cells are exposed to various concentrations of a test MWCNT preparation, cells lysed, and total protein extracts probed with p-IRF3-specific antibody, and optionally IRF3-specific antibody, using any standard immunoassay format (e.g., immunoblot). Typically, the results are compared to parallel assays on untreated cells. As the results presented in Example 5 show, cells exposed to MWCNT preparations respond by phosphorylating/activating IRF3, and the level of p-IRF3 induction appears to be dose dependent.

Phosphorylation of the inactive cytoplasmic form of IRF3 results in the formation of an activated complex between p-IRF3 and CREBBP (CREB binding protein), which translocates to the nucleus and activates the transcription of INFα and IFNβ, as well as other interferon-induced genes, including CCL5. IFNβ and CCL5 are associated with the innate immune response to viral infection. Therefore, by inducing the phosphorylation of IRF3, MWCNTs are able to initiate the IRF3-mediated signal transduction pathway of the innate immune system. MWCNT preparations that have the capacity to induce this response may be characterized as inappropriate for use in certain products of manufacture, and/or exposure to such MWCNTs may be deemed dangerous to individuals working with these materials. Conversely, situations in which an IFR3 activation characteristic may be desirable in MWCNTs can be envisioned, and include, for example, medical treatment protocols in which a therapeutic objective is activation of the innate anti-viral immune response.

Other embodiments of this aspect of the invention include, for example, assays which measure induction of the core cytokine panel, described supra, either alone or in combination with one or more of the additional cytokines whose expression is upregulated by MWCNTs, such as IFNβ.

Various aspects of the invention are further described and illustrated by way of the several examples which follow, none of which are intended to limit the scope of the invention.

### EXAMPLES

### EXAMPLE 1

### MWCNT-Induced Cytokine Protein Expression Profile in Mouse Macrophage-Like Cells

In an effort to identify biomarkers for exposure to carbon-based nanotubes and to identify novel mechanisms of bioactivity, the mouse macrophage-like RAW264.7 cells were used for MWCNT exposure experiments. Macrophages are phagocytic cells that recognize pathogenic organisms and mediate both innate and cell-mediated immune function¹⁵. Macrophage cells represent a first line of defense against immune system challenges and are activated by receptors that bind to pathogen associate molecular patterns (PAMPs). When activated, macrophage cells produce and secrete various chemokines/cytokines that induce both intra- and extracellular signaling to facilitate the host immune response¹⁶. It is conceivable that macrophage cells are involved in recognition of carbon nanotubes and are specifically activated by this type of nanomaterials.

### Materials and Methods:

The MWCNTs used in these experiments were obtained from BuckyUSA (Houston, Tex.) and are >95% MWCNTs of 20-30 nm in diameter and 5-7 μm in length, according to the manufacturers characterization. These MWCNTs were grown using an arc discharge method and may contain trace amorphous carbon, according to the supplier.

To identify biomarkers for MWCNT exposure and to determine if MWCNTs harbor specific bioactivity, RAW264.7 cells were exposed to MWCNTs and the extracellular concentrations of 14 chemokines/cytokines were determined. RAW264.7 cells were exposed to 50 and 500 μg/mL suspensions of MWCNTs for 24 hours. MWCNT suspensions were prepared by sonication of MWCNTs in cell growth media. After 24 h exposure, cell culture supernatants were collected and analyzed to determine the concentration of extracellular cytokines using the Q-Plex Mouse Cytokine Array (Quansys Biosciences, Logan, Utah).

### Results:

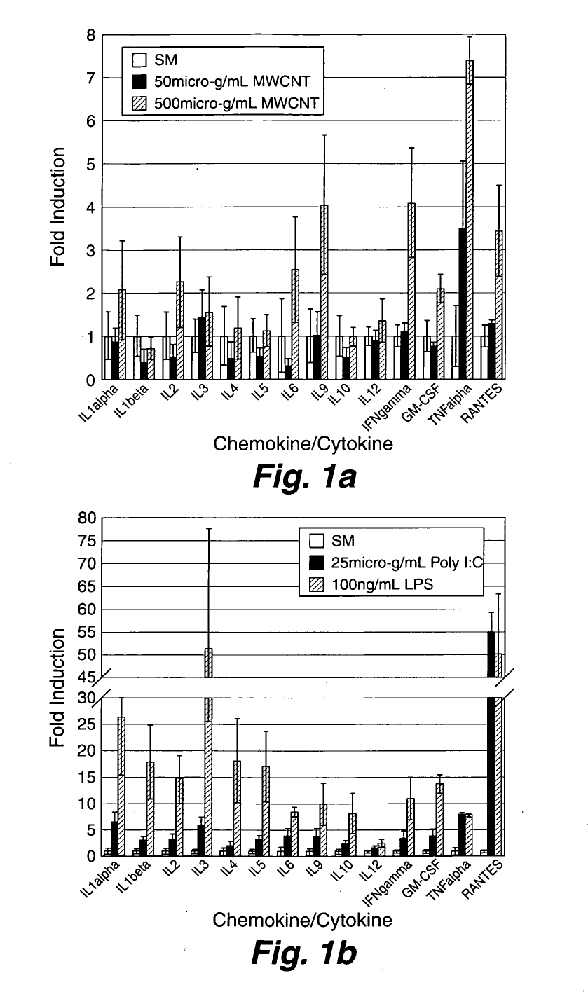

RAW264.7 cell exposure to MWCNTs resulted in at least a 2-fold increase of 8 of the 14 chemokines/cytokines tested when compared to cells exposed to sonicated cell growth media alone (FIG. 1a). The chemokines/cytokines that were elevated upon exposure were Interleukin IL-1α, IL-2, IL-6, IL-9, IFNγ, GM-CSF, TNFα, and RANTES (CCL5).

As a positive control for macrophage cell activation, RAW264.7 cells were exposed to polyinosinic:polycytidylic acid (Poly I:C) or lipopolysaccaride (LPS) using a dose of 25 μg/mL and 100 ng/mL, respectively. Poly I:C and LPS are commonly used as PAMPs for activating an innate immune response. Exposure of RAW264.7 cells to Poly I:C or LPS resulted in increase in the extracellular concentrations for all chemokines/cytokines tested when compared to exposure to sonicated cell growth media (FIG. 1b). Unlike Poly I:C and LPS exposure, which increased the levels of all chemokines/cytokines tested, MWCNT exposure increased the levels of only a subset of these chemokines/cytokines (FIG. 1).

These data suggest that MWCNT exposure results in the induction of a specific set of chemokines/cytokines that may represent a biomarker profile for MWCNT exposure.

### EXAMPLE 2

### Core Cytokine Profile Induced By Multiple MWCNT Preparations

Commercially available MWCNTs can vary as to purity, dimensions, and methods of growth. To determine if the observed RAW264.7 cell activation and subsequent chemokine/cytokine biomarker induction observed in the study described in Example 1, supra, was specific to the MWCNTs obtained from BuckyUSA, RAW264.7 cell activation and chemokine/cytokine biomarker induction was determined in MWCNT exposure experiments using two independent preparations of MWCNTs.

### Materials and Methods:

The alternate preparations were obtained from NanoAmor (Los Alamos, N.M.), and included a “small” MWCNT (sMWCNT, cat #1213NMGS) and an “alternate” MWCNT (aMWCNT, cat # 1234NMG). Both MWCNT preparations were grown using the catalytic chemical vapor deposition (CVD) method and were chosen based on their dimensions. The characterization performed by the supplier showed the following physical characteristics: the sMWCNT preparation consisted of >95-98% MWCNTs 10-30 nm diameter and 1-2 μm length and the aMWCNT preparation consisted of >95-98% MWCNTs 60-100 nm diameter and 5-15 μm length.

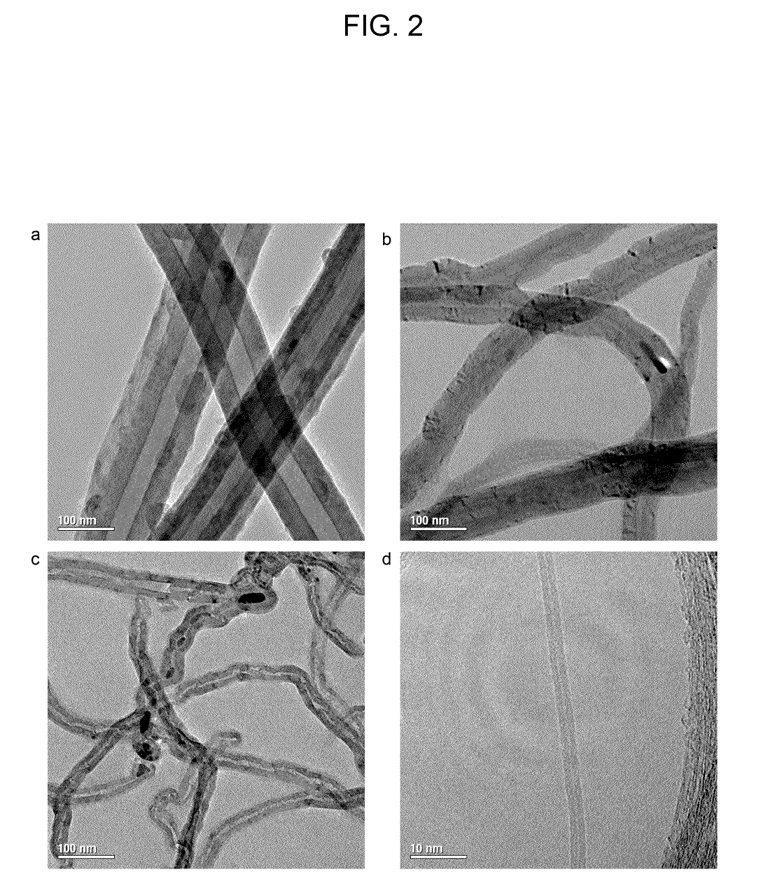

To validate the physical characterization provided by the suppliers, MWCNTs were analyzed using electron microscopy. TEM analysis was performed on the sonicated MWCNT suspensions that were used in the RAW264.7 exposure experiments after placing on a TEM grid and evaporation of the cell growth media. TEM images indicated the presence and integrity of MWCNTs in the cell growth media suspensions (FIG. 2). While length was not determined in these experiments, TEM analysis verified the diameter of the sMWCNTs and aMWCNTs. The MWCNTs from BuckyUSA used in the experiments described in Example 1 were observed to be >100 nm in diameter while the supplier reported a diameter of 20-30 nm. Additionally, single-walled carbon nanotubes were analyzed by TEM (FIG. 2).

To determine if exposure to the different MWCNT preparations results in the induction of the chemokine/cytokine biomarkers identified in the initial exposure experiments, RAW264.7 cells were exposed to MWCNT, sMWCNT, and aMWCNT suspensions prepared, as previously described in Example 1, supra. After 24 h of exposure, cell culture supernatants were collected and the concentrations of extracellular chemokines/cytokines were determined using Q-Plex Mouse Cytokine Array (Quansys Biosciences, Logan, Utah).

### Results:

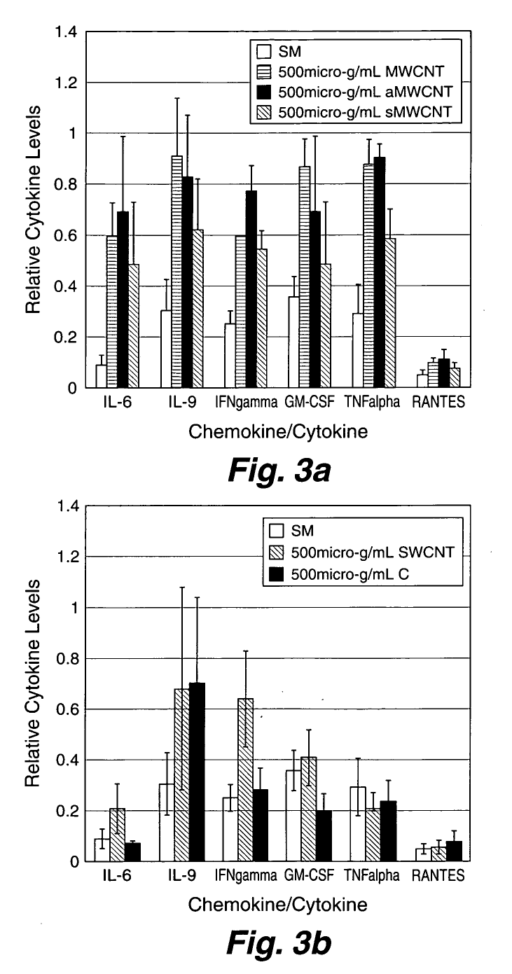

Similar to the observations made in the studies described in Example 1, exposure to aMWCNTs and sMWCNTs resulted in increased levels of extracellular IL-6, IL-9, IFNγ, TNFα and GM-CSF when compared to RAW264.7 cells exposed to sonicated cell growth media only. RANTES levels were also increased upon exposure to all MWCNTs tested, however, this increase was much less than previously observed (FIG. 3a), with the observed induction being 2-fold for MWCNTs, 2.2-fold for aMWCNTs and 1.5-fold for sMWCNTs.

Additionally, the induction of extracellular levels of IL-1α was 1.5-fold, 1.8-fold, and 1.9-fold for MWCNTs, aMWCNTs, and sMWCNTs, respectively. This level of induction was less than 2-fold for all MWCNTs tested. Data represent chemokine/cytokine levels that are normalized to the concentration observed after Poly (I:C) treatment to account for varying levels of RAW264.7 cell activation between experiments. These data indicated that regardless of supplier or size, MWCNT exposure results in the induction of the same biomarker profile.

### EXAMPLE 3

### Specificity of MWCNT-Induced Cytokine Profile

To determine if the observed MWCNT exposure biomarker profile is unique to MWCNTs or if the induction of this biomarker profile results from exposure to other carbon-based nanomaterials, RAW264.7 cells were exposed to 500 μg/mL SWCNTs or 500 μg/mL fullerene suspensions prepared by sonicating in cell growth media. After 24 h of treatment, cell culture supernatants were collected and the concentrations of extracellular chemokines/cytokines were determined as previously described in Example 1, supra.

Exposure to SWCNTs or fullerenes did not result in the induction of the observed MWCNT exposure biomarker profile. However, IL-9 was increased by SWCNT or fullerene exposure and IFNγ was increased in the presence of SWCNTs (FIG. 3b). No specific biomarker profile was observed upon exposure to SWCNTs or fullerenes. The SWCNTs, similar to the MWCNTs used in exposure experiments, contain residual catalyst and amorphous carbon according to the manufacturer. The near absence of RAW264.7 cell activation upon exposure to SWCNTs and fullerenes, as indicated by low levels of chemokine/cytokine induction (FIG. 3b), suggests that the induction of the biomarker profile upon MWCNT exposure is due to the nanotubes themselves, and not to any amorphous carbon or residual catalyst exposure. These data suggested that the biomarker profile observed upon MWCNT exposure is unique to MWCNTs and is not a general characteristic of carbon-based nanomaterials.

### EXAMPLE 4

### MWCNT-Induced Upregulated IFNβ Expression

Increased production and secretion of IL-6, IFNγ, TNFα, GM-CSF and RANTES is associated with the innate immune response to viral infection¹⁷⁻¹⁹. To determine if MWCNT exposure is activating an antiviral cell signaling pathway in RAW264.7 cells, the concentration of extracellular interferon beta was determined following MWCNT exposure using nanotubes from BuckyUSA. The induction of IFNβ, a type-I interferon, is an early event in the innate immune response to viral infection and is the quintessential antiviral cytokine²⁰ .

### Materials and Methods:

Cell culture supernatants from Poly I:C treated cells or cells exposed to 5, 50, and 500 μg/mL suspensions of MWCNTs for 24 h were collected and the concentration of extracellular IFNβ was determined by enzyme-linked immunosorbent assay (ELISA) following the manufacturer protocols (PBL Biomedical Laboratories, Piscataway, N.J.).

### Results:

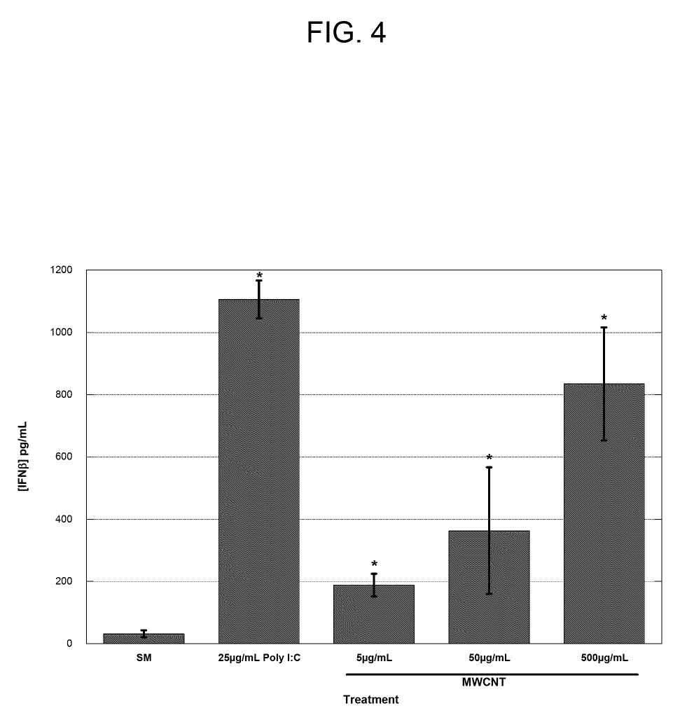

Extracellular IFNβ levels were increased in response to MWCNT exposure in a dose dependent manner (FIG. 4). These data indicated that, similar to the previously identified biomarker chemokines/cytokines, extracellular IFNβ levels were increased upon MWCNT exposure. Furthermore, these data suggest that in the presence of MWCNTs, RAW264.7 cells produce and secrete chemokines/cytokines that are consistent with an antiviral innate immune response.

### EXAMPLE 5

### MWCNT-Induced Accumulation of p-IRF3

IRF3 activation can be observed via the accumulation of the phosphorylated species of IRF3 (p-IRF3) in whole cell protein extracts. This Example evaluates the ability of MWCNTs to induce IRF3 activation using an in vitro immunoassay.

### Materials and Methods:

To determine if IRF3 activation is part of a molecular mechanism of MWCNT bioactivity and biomarker induction, the levels of phospho-IRF3 were determined in RAW264.7 cells exposed to MWCNTs by immunoblot analysis using a p-IRF3 specific antibody (Cell Signaling Technology, Danvers, Mass.)²³.

### Results:

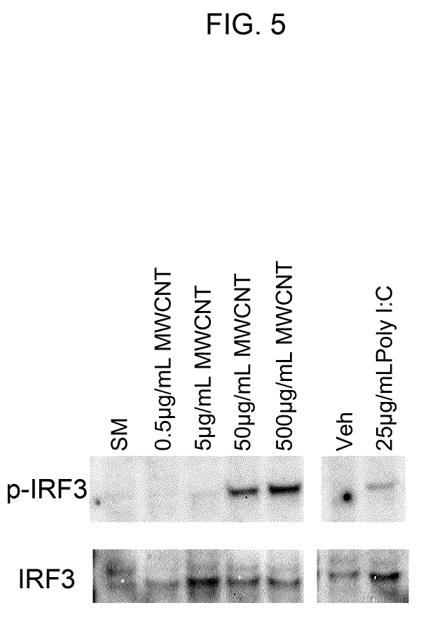

Immunoblot analysis of whole cell protein extracts collected from RAW264.7 cells exposed to various concentrations of MWCNTs indicated that MWCNT exposure leads to the accumulation of p-IRF3 by 3 hrs after treatment in a dose-dependent manner (FIG. 5). These data suggest that IRF3 is activated in RAW264.7 cells upon MWCNT exposure.

### LITERATURE CITED

1. Duguet, E.; Vasseur, S.; Mornet, S.; Devoisselle, J. M. Nanomed 2006, 1, (2), 157-68.
2. Kagan, V. E.; Bayir, H.; Shvedova, A. A. Nanomedicine 2005, 1, (4), 313-6.
3. Kumar, S.; Richards-Kortum, R. Nanomed 2006, 1, (1), 23-30.
4. Gur, I.; Fromer, N. A.; Geier, M. L.; Alivisatos, A. P. Science 2005, 310, (5747), 462-5.
5. Bianco, A.; Kostarelos, K.; Prato, M. Curr Opin Chem Biol 2005, 9, (6), 674-9.
6. Monteiro-Riviere, N. A.; Inman, A. O.; Wang, Y. Y.; Nemanich, R. J. Nanomedicine. 2005, 1, (4), 293-9.
7. Shvedova, A. A.; Castranova, V.; Kisin, E. R.; Schwegler-Berry, D.; Murray, A. R.; Gandelsman, V. Z.; Maynard, A.; Baron, P. J Toxicol Environ Health A 2003, 66, (20), 1909-26.
8. Murr, L. E.; Garza, K. M.; Soto, K. F.; Carrasco, A.; Powell, T. G.; Ramirez, D. A.; Guerrero, P. A.; Lopez, D. A.; Venzor, J., 3rd. Int J Environ Res Public Health 2005, 2, (1), 31-42.
9. Ding, L.; Stilwell, J.; Zhang, T.; Elboudwarej, O.; Jiang, H.; Selegue, J. P.; Cooke, P. A.; Gray, J. W.; Chen, F. F. Nano Lett 2005, 5, (12), 2448-64.
10. Pulskamp, K.; Diabate, S.; Krug, H. F. Toxicol Lett 2007, 168, (1), 58-74.
11. Kagan, V. E.; Tyurina, Y. Y.; Tyurin, V. A.; Konduru, N. V.; Potapovich, A. I.; Osipov, A. N.; Kisin, E. R.; Schwegler-Berry, D.; Mercer, R.; Castranova, V.; Shvedova, A. A. Toxicol Lett 2006, 165, (1), 88-100.
12. Sato, Y.; Yokoyama, A.; Shibata, K.; Akimoto, Y.; Ogino, S.; Nodasaka, Y.; Kohgo, T.; Tamura, K.; Akasaka, T.; Uo, M.; Motomiya, K.; Jeyadevan, B.; Ishiguro, M.; Hatakeyama, R.; Watari, F.; Tohji, K. Mol Biosyst 2005, 1, (2), 176-82.
13. Worle-Knirsch, J. M.; Pulskamp, K.; Krug, H. F. Nano Lett 2006, 6, (6), 1261-8.
14. Murr, L. E.; Esquivel, E. V.; Bang, J. J.; de la Rosa, G.; Gardea-Torresdey, J. L. Water Res 2004, 38, (19), 4282-96.
15. Morrissette, N.; Gold, E.; Aderem, A. Trends Cell Biol 1999, 9, (5), 199-201.
16. Medzhitov, R.; Janeway, C., Jr. N Engl J Med 2000, 343, (5), 338-44.
17. Julkunen, I.; Melen, K.; Nyqvist, M.; Pirhonen, J.; Sareneva, T.; Matikainen, S. Vaccine 2000, 19 Suppl 1, S32-7.
18. Douville, R. N.; Bastien, N.; Li, Y.; Pochard, P.; Simons, F. E.; HayGlass, K. T. J Immunol 2006, 176, (10), 5848-55.
19. Molina, J. M.; Schindler, R.; Ferriani, R.; Sakaguchi, M.; Vannier, E.; Dinarello, C. A.; Groopman, J. E. J Infect Dis 1990, 161, (5), 888-93.
20. Zuniga, E. I.; Hahm, B.; Oldstone, M. B. Curr Top Microbiol Immunol 2007, 316, 337-57.
21. Juang, Y. T.; Lowther, W.; Kellum, M.; Au, W. C.; Lin, R.; Hiscott, J.; Pitha, P. M. Proc Natl Acad Sci U S A 1998, 95, (17), 9837-42.
22. Lin, R.; Heylbroeck, C.; Genin, P.; Pitha, P. M.; Hiscott, J. Mol Cell Biol 1999, 19, (2), 959-66.
23. Lin, R.; Heylbroeck, C.; Pitha, P. M.; Hiscott, J. Mol Cell Biol 1998, 18, (5), 2986-96.

What is claimed is:

1. An assay for evaluating MWCNT exposure in an individual, comprising:
(a) determining the expression levels of the cytokines IL-6, IL-9, IFNγ, TNFα and GM-CSF in a test biological sample obtained from the individual; and,
(b) comparing the expression levels so determined to the expression levels in a corresponding normal sample,
wherein the presence of significantly elevated expression levels of each of the cytokines IL-6, IL-9, IFNγ, TNFα and GM-CSF in the test sample relative to the normal sample provides an indication of MWCNT exposure.

2. The assay of claim 1, further comprising determining the expression levels of one or more of the cytokines IL-1α, IL2, IL3, IFNβ and CCL5 in both the test and normal samples, wherein the presence of significantly elevated expression levels of each of the cytokines IL-6, IL-9, IFNγ, TNFα and GM-CSF, plus significantly elevated expression levels in one or more of the cytokines IL-1α, IL2, IL3, IFNβ and CCL5, in the test sample relative to the normal sample, provides an indication of MWCNT exposure.

3. The assay of claim 1 or 2, further comprising detecting the presence of phosphorylated IRF3 protein in the test sample.

4. An assay for evaluating MWCNT exposure in an individual, comprising detecting the presence of phosphorylated IRF3 protein in a biological sample from the individual.

5. The assay of claim 1 or 2, wherein the expression levels of the cytokines are determined at the protein level.

6. The assay of claim 1 or 2, wherein wherein the expression levels of the cytokines are determined at the mRNA level.

7. The assay of claim 1 or 2, wherein the biological test sample is peripheral blood, serum, or a cellular fraction thereof.

8. An in vitro assay for evaluating whether a MWCNT preparation is capable of initiating the IRF3-mediated signal transduction pathway of the innate immune system in mammalian cells, comprising:
(a) exposing cultured mammalian cells to the MWCNT preparation;
(b) extracting total protein from the cells;
(c) detecting phosphorylated IRF3 protein in the extracted total protein, wherein the detection of phosphorylated IRF3 protein in the extracted total protein provides an indication that the MWCNT preparation is capable of initiating the IRF3-mediated signal transduction pathway of the innate immune system.

9. The assay of claim 8, wherein detecting phosphorylated IRF3 is carried out with an immunoassay utilizing an antibody specific for phosphorylated IRF3.

10. The assay of claim 9, wherein the mammalian cells are monocytes or macrophages.
